(19) United States
(12) Patent Application Publication
(10) Pub. No.: US 2008/0249192 A1
(43) Pub. Date: Oct. 9, 2008

(54) Wax Mixture Based on Partial Glycerides and Pentaerythriol Esters

(76) Inventors: Caroline Goget, Paris (FR); Ulrich Issberner, Ambler, PA (US); Rolf Kawa, Monheim (DE); Catherine Weichold, Aachen (DE)

Correspondence Address: SYNNESTVEDT & LECHNER LLP, 1101 MARKET STREET, PHILADELPHIA, PA, 19107, US

(21) Appl. No.: 11/547,592

(22) PCT Filed: Mar. 26, 2005
(86) PCT No.: PCT/EP05/03233

§ 371 Date: Mar. 17, 2008

(30) Foreign Application Priority Data
Apr. 5, 2004 (DE) 10 2004 017 222.6
Jun. 30, 2004 (DE) 10 2004 031 939.1
Aug. 14, 2004 (DE) 10 2004 039 471.7

(51) Int. Cl. A61K 47/14

(52) U.S. Cl. 514/786

(57) ABSTRACT

A wax mixture containing esters of at least one of pentaerythritol, dipentaerythritol, an tripentaerythritol and partial glycerides and a self-emulsifying base containing the wax mixture and an emulsifier which can be a nonionic emulsifier, anionic emulsifier and mixtures of the emulsifiers with an HLB value of at least 10.

### FIELD OF THE INVENTION

This invention relates to wax mixtures of partial glycerides and esters of pentaerythritol or oligomers of pentaerythritol and to self-emulsifying mixtures based on the wax mixtures. The invention also relates to compositions based on the self-emulsifying mixtures.

### PRIOR ART

Wax-based formulations have long been known to the expert and are used inter alia for cosmetic and pharmaceutical formulations, such as suppositories for example, various cosmetic compositions in stick form or in creams and lotions, for coating papers and textiles, etc. Numerous wax-like substances and mixtures of such substances are available to the expert for this purpose. These include, for example, glycerides and fatty alcohols which have a critical effect on the sensory profile of the final formulations. Thus, glycerides leave the skin with an often oily/greasy feeling. Fatty alcohols lead to the formation of white residues which users find to be a major disadvantage. In addition, fatty alcohols often lead to a marked rise in viscosity in storage.

Waxes and combinations of waxes are also used inter alia for favorably influencing the consistency and viscosity of cosmetic formulations. The viscosity of cosmetic emulsions, particularly oil-in-water (o/w) emulsions, is generally adjusted with so-called hydrophilic waxes. These are substances or mixtures of substances which have a melting point above 30° C. and which contain free OH groups in the molecule that are capable of interacting with water (hydrate formation by hydrogen bridge bond) and hence make a considerable contribution to the buildup of viscosity in o/w emulsions through the formation of so-called lamellar phases. Viscosity not only influences phase stability, it also has a clearly positive effect on key sensory parameters, including inter alia cushion, distribution and peaking. The cosmetic chemist resorts to such waxes in order to adapt emulsions exactly to one another in regard to sensory impression and viscosity. The disadvantage of lamellar phases is that they are not resistant to ageing, so that the viscosities can decrease or increase as a function of time with the result that, ultimately, the sensory impression is affected. Although a solution to the problem of obtaining stable viscosities is disclosed in DE 31 31 006 A1, which uses a carefully balanced wax combination of partial glycerides, fatty alcohols, wax esters and triglycerides, this solution always fails when the predetermined quantity ratios are slightly varied, as stated in the document in question.

The problem addressed by the present invention was to provide combinations of known hydrophilic waxes which would guarantee durably stable viscosities of o/w emulsions over a broad mixing ratio, irrespective of the structure of the emulsifiers used and the polarity of the emollients used.

It has now surprisingly been found that this problem can be solved by specific wax mixtures based on esters of pentaerythritol or oligomers thereof in combination with partial glycerides.

### DESCRIPTION OF THE INVENTION

Accordingly, the present invention relates to water-free wax mixtures containing at least one ester of pentaerythritol, dipentaerythritol, tripentaerythritol or a mixture of these esters as component (a) and at least one partial glyceride as component (b), the ratio by weight of (a) to (b) being from 1:3 to 3:1.

Water-free wax mixtures in the context of the invention are wax mixtures which contain less than 2% by weight water, preferably less than 1% by weight water, more preferably less than 0.5% by weight water and most preferably less than 0.1% by weight water. The wax mixtures normally only contain residues of water emanating from the raw materials used.

It has surprisingly been found that o/w emulsions containing the wax mixtures according to the invention not only have “stable” viscosities for several weeks, the viscosities of the emulsions are also significantly higher than the viscosities obtained using the same quantity of a single wax component. In addition, the buildup of viscosity in emulsions based on the wax mixtures according to the invention is independent of the polarity of the oils used and, hence, independent of the structure of the emulsifiers used. The wax mixtures according to the invention allow the cosmetic chemist to achieve an exact balance between sensory impression and viscosity without any danger of the sensory properties being adversely affected by changes in viscosity as a function of time.

### Component (a)

According to the invention, preferred wax mixtures are characterized in that component (a) is selected from the group of C₆₋₂₂ fatty acid esters of pentaerythritol, dipentaerythritol, tripentaerythritol or mixtures of these esters which have a melting point of at least 30° C.

The esters may contain a single type of fatty acid acyl groups or a mixture of various fatty acid acyl groups; the fatty acids may be branched or unbranched and/or saturated or unsaturated. Fatty acids/fatty acid mixtures with a large content of saturated unbranched fatty acids, particularly those emanating from vegetable raw material sources, are preferably used for the esterification. According to the invention, C₁₄₋₂₄ fatty acids and particularly C₁₄₋₂₀ fatty acids are preferred. These include, for example, myristic, pentadecanoic, palmitic, margaric, stearic, nonadecanoic, arachic, behenic, lignoceric, cerotic, melissic, erucic and elaeostearic acid and substituted fatty acids such as, for example, 12-hydroxystearic acid.

Another preferred embodiment of the wax mixture is characterized in that component (a) is selected from the group of esters of pentaerythritol with a percentage content of (i) 5 to 35% by weight monoesters, (ii) 20 to 50% by weight diesters and (iii) 25 to 50% by weight triesters and optionally tetraesters. A content of (a) 10 to 25% by weight monoesters, (b) 25 to 40% by weight diesters and (c) 30 to 45% by weight triesters and optionally tetraesters is preferred while a content of (a) 12 to 19% by weight monoesters, (b) 25 to 35% by weight diesters, (c) 30 to 40% by weight triesters and (d) 6 to 11% by weight tetraesters is most particularly preferred. A most particularly preferred embodiment of the wax mixture contains esters of pentaerythritol, dipentaerythritol or tripentaerythritol which contain less than 0.3% by weight C₁₇ fatty acyl groups as the ester component (a).

Another preferred embodiment of the wax mixture is characterized in that the ester component (a) is obtained by esterification with a fatty acid mixture containing 40 to 50% by weight C₁₆ fatty acid and 45 to 55% by weight C₁₈ fatty acid. The rest of the fatty acid mixture is made up of relatively short-chain (≦C₁₄) and relatively long-chain (>C₁₈) fatty acids. Esters of pentaerythritol and dipentaerythritol with a ratio by weight of C₁₆/C₁₈ fatty acids of ca. 0.7 to 0.9 are superior in terms of sensory properties. According to the invention, a particularly suitable ester is an ester of pentaerythritol which is obtained by reaction of pentaerythritol with a fatty acid mixture containing 42 to 48% by weight C₁₆ fatty acid and 50 to 56% by weight C₁₈ fatty acid (rest ≦C₁₄ fatty acids and > C₁₈ fatty acids) and which has the following ester distribution: 12 to 19% by weight monoesters, (b) 25 to 35% by weight diesters, (c) 30 to 40% by weight triesters and (d) 6 to 11% by weight tetraesters. Normally, 1.8 to 2.2 mol and, preferably, 1.9 to 2.1 mol of the fatty acid mixture per mol pentaerythritol is used for the esterification.

C₁₆/C₁₈ fatty acid pentaerythritol esters can be produced, for example, by a process in which 1.8 to 2.2 mol and, preferably, 1.9 to 2.1 mol per mol pentaerythritol of a fatty acid mixture containing 40 to 50% by weight C₁₆ fatty acid and 45 to 55% by weight C₁₈ fatty acid or a raw material mixture with a corresponding fatty acid distribution is used and (a) the esterification is carried out at temperatures of 180° C. to 250° C. in an inert gas atmosphere in the absence of solvent, (b) the water formed is distilled off, (c) the reaction mixture obtained is stirred in vacuo until it has an acid value of <1 and an OH value of 145 to 158, (d) unreacted pentaerythritol is filtered off and (e) an aftertreatment with hydrogen peroxide is optionally carried out. Methods for monitoring and adjusting acid value and OH value are well-known to the expert, so that there is no need to discuss them in detail here.

### Component (b)

A preferred embodiment of the wax mixture is characterized in that the partial glyceride (b) is selected from the group of esterification products of glycerol or oligoglycerol with C₁₂₋₂₄ fatty acids or mixtures of these esterification products. Oligoglycerols in the context of the invention are oligomers of glycerol containing 2 to 12 glycerol units.

Fatty acids suitable for the esterification include, for example, myristic, pentadecanoic, palmitic, margaric, stearic, nonadecanoic, arachic, behenic, lignoceric, cerotic, melissic, erucic and elaeostearic acid and substituted fatty acids, such as 12-hydroxystearic acid for example, this list being of a purely exemplary, non-limiting character. A preferred embodiment of the invention is characterized by the use of fatty acids/fatty acid mixtures which have a high percentage content of unbranched, saturated fatty acids.

The partial glyceride (b) is preferably a palmitic and/or stearic acid ester of glycerol with a monoglyceride content of 30 to 65% by weight and a diglyceride content of 25 to 45% by weight. With a monoglyceride content of at least 30% by weight, the buildup of viscosity is guaranteed particularly effectively and the final cosmetic formulations are distinguished by improved long-term stability. In addition, up to an upper limit of 65% by weight monoglyceride, the danger of crystallizing out is fairly minimal.

The partial glyceride (b) is preferably obtained by reaction of glycerol with a fatty acid mixture of palmitic and stearic acid in a ratio by weight of 1:3 to 3:1.

Partial glycerides suitable for use in accordance with the invention include, for example, the products Cutina® MD (30-45% by weight monoglyceride content/diglyceride content 35-45% by weight), Cutina® GMS (40-55% by weight monoglyceride content/diglyceride content 30-45% by weight) and Monomuls 60-35 (55-65% by weight monoglyceride content/diglyceride content 25-45% by weight) marketed by Cognis Deutschland GmbH & Co. KG.

Mixed esters and mixtures of mono-, di- and triglycerides are particularly suitable for the purposes of the invention because they have a relatively low tendency towards crystallization and thus improve the performance of the composition according to the invention. In addition, they show considerably better compatibility with oils of widely varying polarity.

In the formulation of cosmetic or pharmaceutical compositions, the wax mixtures according to the invention are normally processed with oil components, water and emulsifiers. From the application perspective, it is of particular advantage to provide a self-emulsifying base based on the wax mixtures according to the invention which already contains the necessary quantity of emulsifiers.

### Self-Emulsifying Base

Accordingly, the present invention relates to self-emulsifying bases containing the wax mixture claimed in any of claims 1 to 7 and, in addition, at least one emulsifier (c) selected from the group of anionic or nonionic emulsifiers with an HLB value of greater than 10. In the context of the present invention, there is no difference between the terms “emulsifier” and “surfactant”.

### Emulsifiers (c)

As mentioned above, the structure of the emulsifiers is not particularly critical. Detailed lists of the HLB values of commercially available emulsifiers are known to the expert and can be found, for example, in Fiedler, Lexikon der Hilfsstoffe für Pharmazie, Kosmetik und angrenzende Gebiete, Vol. 9, 1971, pages 265-270; Kirk-Othmer (3^{rd} Edition), Vol. 8, pages 909-918 and Janistyn (3^{rd} Edition), Vol. 1, page 470; and Vol. 3, pages 68-78. The anionic and nonionic emulsifiers with an HLB value above 10 listed there are intended to be part of the present disclosure. The emulsifiers may be used as the active substance or in the form of aqueous solutions. Examples of anionic emulsifiers/surfactants which may be used in accordance with the invention are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl phosphates and alkyl ether phosphates. If the anionic surfactants contain polyglycol ether chains, they may have a conventional homolog distribution although they preferably have a narrow-range homolog distribution.

The group of particularly suitable nonionic emulsifiers/surfactants with an HLB value > 10 includes corresponding compounds selected from
(1) products of the addition of ethylene oxide and/or propylene oxide onto linear and branched C₈₋₄₀ fatty alcohols, onto linear and branched C₁₂₋₄₀ fatty acids and onto alkylphenols with 8 to 15 carbon atoms in the alkyl group;
(2) C₁₂₋₁₈ fatty acid mono- and diesters of addition products of ethylene oxide and/or propylene oxide onto glycerol;
(3) glycerol mono- and diesters and sorbitan mono- and diesters of saturated and unsaturated C₆₋₂₂ fatty acids and ethylene oxide and/or propylene oxide addition products thereof;
(4) alkyl mono- and oligoglycosides with 8 to 22 carbon atoms in the alkyl chain and ethoxylated analogs thereof;
(5) products of the addition of ethylene oxide and/or propylene oxide onto castor oil and/or hydrogenated castor oil.

Individual examples (cf. Kirk-Othmer) of nonionic emulsifiers/surfactants with an HLB value of at least 10 are POE (5) sorbitan monooleate (POE=polyoxyethylene), POE (40) sorbitol hexaoleate, PEG 400 dilaurate, POE (5) nonylphenol(ether), POE (20) sorbitan tristearate, POP/POE condensate, POE (6) nonylphenol(ether), POE (20) lanolin (ether und ester), POE (20) sorbitan trioleate, POE (8) stearic acid (monoester), POE (50) sorbitol hexaoleate, POE (6) tridecyl alcohol (ether), PEG 400 monostearate, POE (8) nonylphenol (ether), POE (10) stearyl alcohol (ether), POE (8) tridecyl alcohol (ether), POE (8) lauric acid (monoester), POE (10) cetylalcohol (ether), acetylated POE (10) lanolin, POE (20) glycerol monostearate, PEG 400 monolaurate, POE (16) lanolin alcohol (ether), POE (4) sorbitan monolaurate, POE (10) nonylphenol (ether), POE (15) long oil fatty acids (esters), POE (10) octylphenol (ether), PEG 600 monostearate, tertiary amines: POE fatty amines; POE (24) cholesterol, POE (14) nonylphenol (ether), POE (12) laurylalcohol, POE (20) sorbitan monostearate, sucrose monolaurate, POE (20) sorbitan monooleate, acetylated POE (9) lanolin, POE (20) stearyl alcohol, POE (20) oleyl alcohol (ether), PEG 1000 monooleate, POE (20) tallow amine, POE (20) sorbitan monopalmitate, POE (20) cetyl alcohol (ether), POE (25) propylene glycol monostearate, POE (20) nonylphenol (ether), PEG (1000) monolaurate, POE (20) sorbitan monolaurate, POE (23) lauryl alcohol (ether), POE (40) stearic acid (monoester), POE (50) lanolin (ether and ester), POE (25) soya sterol, POE (30) nonylphenol (ether), PEG 4000 distearate, POE (50) stearic acid (monoester), POE (70) dinonylphenol (ether), POE (20) castor oil (ether, ester), N-cetyl-N-ethyl-morpholinium ethyl sulfate, etc.

The self-emulsifying bases enable viscosity-stable o/w emulsions to be produced particularly easily because the ratio of the emulsifiers to the consistency-imparting waxes is already exactly balanced.

According to the invention, it is of particular advantage to use a self-emulsifying base containing
(a) 20 to 60% by weight of at least one ester of pentaerythritol, dipentaerythritol and/or tripentaerythritol according to any of claims 2 to 5,
(b) 20 to 60% by weight of at least one partial glyceride according to any of claims 6 to 8 and
(c) 5 to 30% by weight and preferably 10 to 30% by weight of at least one additional emulsifier selected from the group of anionic or nonionic emulsifiers with an HLB value above 10,
   the self-emulsifying mixture containing less than 20% by weight water.

The percentages by weight are based on the quantity of active substance in the self-emulsifying composition. A water content of less than 15% by weight is preferred, a water content of less than 10% by weight being particularly preferred.

A preferred embodiment of the self-emulsifying base is characterized in that the anionic emulsifier is selected from the alkali metal salts of C₁₂₋₁₄ acyl glutamates, from the alkali metal and triethanolamine salts of C₁₂₋₂₄ fatty acids and from the alkali metal salts of C₁₂₋₂₄ fatty alcohol sulfates/C₁₂₋₂₄ fatty alcohol ether sulfates or C₁₂₋₂₄ fatty alcohol phosphates/C₁₂₋₂₄ fatty alcohol ether phosphates. The alkali metal salts of C₁₋₂₄ acyl glutamates, the alkali metal and triethanolamine salts of C₁₆₋₂₄ fatty acids and the alkali metal salts of C₁₆₋₂₄ fatty alcohol (ether) sulfates or C₁₆₋₂₄ fatty alcohol (ether) phosphates are particularly preferred. The alkali metal salts of C₁₆₋₁₈ acyl glutamates, the alkali metal and triethanolamine salts of C₁₆₋₁₈ fatty acids and the alkali metal salts of C₁₆₋₁₈ fatty alcohol (ether) sulfates or C₁₆₋₁₈ fatty alcohol (ether) phosphates are most particularly preferred. According to the invention, monosodium stearoyl glutamate, disodium stearoyl glutamate or triethanolamine, potassium or sodium stearate is most particularly preferred. Preferred acyl glutamates are commercially available, for example, under the name of Amisoft® from Ajinomoto. Amisoft® HS-21 P (Disodium Stearoyl Glutamate) and Amisoft® HS-11 P (Sodium Stearoyl Glutamate) are preferably used.

Soaps particularly suitable for the purposes of the invention contain palmitic and stearic acid in a ratio by weight of 1:3 to 3:1.

Another preferred embodiment of the self-emulsifying base is characterized in that the nonionic emulsifiers with an HLB value above 10 are selected from the group of C₁₂₋₂₄ fatty alcohol ethoxylates and/or C₁₂₋₂₄ alkyl aligoglucosides.

C₁₂₋₂₄ fatty alcohol ethoxylates suitable for use in accordance with the invention include, for example, Ceteareth-12, Ceteareth-20, Ceteareth-30, which are marketed by Cognis Deutschland GmbH & Co. KG under the names of Eumulgin® B1, Eumulgin® B2 and Eumulgin® B3.

The C₁₂₋₂₄ alk(en)yl oligoglycosides preferably used in accordance with the invention are known nonionic surfactants which correspond to formula (I):

R¹-[G]ₚ (I)

where R¹ is a C₁₂₋₂₄ alk(en)yl group, G is a sugar unit containing 5 or 6 carbon atoms and p is a number of 1 to 10. They may be obtained by the relevant methods of preparative organic chemistry. EP-A1-0 301 298 and WO 90/03977 are cited here as representative of the literature abundantly available on the subject.

The alk(en)yl oligoglycosides may be derived from aldoses or ketoses containing 5 or 6 carbon atoms, preferably glucose. Accordingly, the preferred alk(en)yl oligoglycosides are alk(en)yl oligoglucosides. The index p in general formula (I) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alk(en)yl oligoglycoside is an analytically determined calculated quantity which is generally a broken number. Alk(en)yl oligoglycosides having an average degree of oligomerization p of 1.1 to 3.0 are preferably used. Alk(en)yl oligoglycosides having a degree of oligomerization of less than 1.7 and, more particularly, between 1.2 and 1.4 are preferred from the applicational perspective. Alkyl oligoglucosides in which the substituent R¹ is derived from primary, preferably unbranched C₁₂₋₂₄, preferably C₁₆₋₂₄ and more particularly C₁₆₋₁₈ alcohols are preferably used in accordance with the invention. Technical mixtures of the alcohols may also be used. Particularly preferred alkyl oligoglucosides are marketed under the names of Plantacare® 1200, Plantacare® 2000, Plantacare® 810 and Plantacare® 818, Tego Care® CG 90, Emulgade® PL 68/50, Montanov® 14, Montanov® 202, Montanov® 68, Montanov® 82, Montanov® L and Montanov® S, Oramix® BG 14, Oramix® CG 110, Oramix® NS 10.

### Commercial Applications

The wax mixtures according to the invention and the self-emulsifying bases are used for the production of cosmetic and pharmaceutical compositions.

Accordingly, the present invention also relates to cosmetic or pharmaceutical preparations containing 1 to 20% by weight of a wax mixture according to the invention or 1 to 20% by weight of a self-emulsifying base according to the invention. The concentration used is between 2 and 10% by weight in either case. The present invention also relates to the use of the wax mixture claimed in any of claims 1 to 7 for building up viscosity in emulsions, particularly oil-in-water emulsions, and to the use of the self-emulsifying mixture claimed in any of claims 8 to 11 for building up viscosity in emulsions, particularly oil-in-water emulsions. The present invention also relates to the use of the wax mixture claimed in any of claims 1 to 7 or the self-emulsifying mixture claimed in any of claims 8 to 11 as a consistency factor in cosmetic or pharmaceutical emulsions. Depending on the concentration used, lotions or creams are formed and are characterized in that they have an average particle size of 1 μm to 20 μm and preferably 5 μm to 10 μm. The viscosities of such emulsions are in the range from 2,000 mPa·s to 500,000 mPa·s and preferably in the range from 10,000 mPa·s to 200,000 mPa·s. The viscosities were measured with a Brookfield RVF at 23° C. using spindle 5 or 6 at 10 r.p.m. (up to 60,000 mPa·s for lotions) or spindle HT with Helipath (>60,000 mPa·s for creams).

### Oil Components

The preparations according to the invention contain an aqueous phase and an oil phase which may contain auxiliaries and additives. The percentage content of aqueous phase is typically in the range from 20 to 90% by weight, based on the composition as a whole, and the percentage content of oil phase typically in the range from 1 to 70% by weight, based on the composition as a whole. The oil phase may be made up of an oil component or a mixture of oil components. The oil component(s) is/are present in a quantity of 1 to 25% by weight, preferably in a quantity of 1 to 20% by weight and more particularly in a quantity of 5 to 15% by weight, based on the composition as a whole.

Suitable oil components are, for example, the classes of compounds mentioned in the following: Guerbet alcohols based on fatty alcohols containing 6 to 18 and preferably 8 to 10 carbon atoms, esters of linear or branched, saturated or unsaturated C₆₋₂₂ fatty acids with linear or branched, saturated or unsaturated C₆₋₂₂ fatty alcohols, more particularly 2-ethyl hexanol. The following are mentioned by way of example: hexyl laurate, myristyl isostearate, myristyl oleate, cetyl isostearate, cetyl oleate, stearyl isostearate, stearyl oleate, isostearyl myristate, isostearyl palmitate, isostearyl stearate, isostearyl isostearate, isostearyl oleate, oleyl myristate, oleyl isostearate, oleyl oleate, oleyl erucate, erucyl isostearate, erucyl oleate, cococaprylate/caprate. Other suitable esters are, for example, are esters of C₁₈₋₃₈ alkylhydroxycarboxylic acids with linear or branched, saturated or unsaturated C₆₋₂₂ fatty alcohols, esters of linear and/or branched, saturated or unsaturated fatty acids with polyhydric alcohols (for example propylene glycol, dimer diol or trimer triol) and/or Guerbet alcohols, triglycerides or triglyceride mixtures, liquid mono-, di- and triglyceride mixtures, esters of C₆₋₂₂ fatty alcohols and/or Guerbet alcohols with aromatic carboxylic acids, more particularly benzoic acid, esters of C₂₋₁₂ dicarboxylic acids with linear or branched, saturated or unsaturated alcohols containing 1 to 22 carbon atoms or polyols containing 2 to 10 carbon atoms and 2 to 6 hydroxyl groups, vegetable oils, branched primary alcohols, substituted cyclohexanes, linear dialkyl carbonates (for example Cetiol® CC), Guerbet carbonates based on fatty alcohols containing 6 to 18 and preferably 8 to 10 carbon atoms, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Di-n-octyl Ether (Cetiol® OE) or ring opening products of epoxidized fatty acid esters with polyols, hydrocarbons, such as paraffin or mineral oils, oligo- or poly-α-olefins. According to the invention, dialkyl ethers, dialkyl carbonates, triglyceride mixtures and esters of C₈₋₂₄ fatty acids and C₈₋₂₄ fatty alcohols or mixtures of these substances are particularly suitable for use as the oil component. The dialkyl carbonates and dialkyl ethers may be symmetrical or asymmetrical, branched or unbranched, saturated or unsaturated and may be produced by reactions known from the prior art. Suitable silicone compounds are, for example, dimethyl polysiloxanes, methylphenyl polysiloxanes, cyclic silicones (cyclomethicone) and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

According to the invention, other suitable oil components are inter alia hydrocarbons, preferably with a chain length of 8 to 40 carbon atoms. They may be branched or unbranched, saturated or unsaturated. Of these, branched, saturated C₈₋₄₀ alkanes are preferred. Both pure substances and mixtures may be used. The mixtures are normally mixtures of different isomeric compounds. Compositions containing C₁₀₋₃₀, preferably C₁₂₋₂₀ and more particularly C₁₆₋₂₀ alkanes are particularly suitable and, of these, a mixture of alkanes containing at least 10% by weight branched alkanes, based on the total quantity of alkanes, is particularly preferred. The alkanes are preferably branched, saturated alkanes. Mixtures of alkanes containing more than 1% by weight 5,8-diethyl dodecane and/or more than 1% by weight didecene are particularly suitable.

### Other Optional Auxiliaries and Additives

Depending on their intended application, the cosmetic formulations may contain a number of other auxiliaries and additives such as, for example, thickeners, superfatting agents, stabilizers, polymers, lecithins, phospholipids, biogenic agents, UV protection factors, antioxidants, deodorants, film formers, swelling agents, insect repellents, hydrotropes, solubilizers, preservatives, perfume oils, dyes, etc. which are listed by way of example in the following. The quantities in which the particular additives are used is determined by the intended use.

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl and hydroxypropyl cellulose, polyvinyl alcohol, polyvinyl pyrrolidone and bentonites such as, for example, Bentone® GeIVS-5PC (Rheox).

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. Typical UV-A filters are, in particular, derivatives of benzoyl methane. The UV-A and UV-B filters may of course also be used in the form of mixtures, for example combinations of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene), and esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are often combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide. Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin.

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract, bambara nut extract, and vitamin complexes.

Deodorizing components counteract, mask or eliminate body odors. Body odors are formed through the action of skin bacteria on apocrine perspiration which results in the formation of unpleasant-smelling degradation products. Accordingly, suitable deodorizing components are inter alia germ inhibitors, enzyme inhibitors, odor absorbers or odor maskers.

Suitable insect repellents are, for example, N,N-diethyl-m-toluamide, pentane-1,2-diol or 3-(N-n-butyl-N-acetylamino)-propionic acid ethyl ester), which is marketed as Insect Repellent® 3535 by Merck KGaA, and Butylacetylaminopropionate.

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetikverordnung (“Cosmetics Directive”).

Suitable perfume oils are mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, and synthetic perfume compounds of the ester, ether, aldehyde, ketone, alcohol and hydrocarbon type, are also suitable.

Suitable dyes are any of the substances suitable and approved for cosmetic purposes. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). These dyes are normally used in concentrations of 0.001 to 0.1% by weight, based on the mixture as a whole.

The following Examples describe emulsions based on the wax mixtures according to the invention and show the buildup of viscosity and viscosity profile by comparison with emulsions based on single waxes and wax mixtures which do not correspond to the mixing ratio according to the invention.

### EXAMPLES

Production was carried out by the hot method (200 g laboratory batch). To this end, the oil phase was heated to 80° C. The water phase—also heated to 80° C.—was slowly added to the oil phase with stirring. The emulsion was cooled to 40° C. with continuous stirring. If necessary, the pH value was adjusted to pH 7 with citric acid or sodium hydroxide at 40° C. and cooling was continued to 30° C. In the cooling phase between 45 and 65° C., the emulsion was homogenized for 2 minutes with a suitable rotor/stator system (for example IKA Ultra Turrax T 50). The viscosities (Brookfield RVF, spindle 5, 10 r.p.m., 23° C.) of the emulsions thus prepared were determined after 4, 8 and 12 weeks (W) and are compared in the following Tables. The viscosity measurements were carried out at room temperature.

The quantities shown in the following Tables represent % by weight of the commercially available substance in the composition as a whole. The Example Nos. preceded by the letter “C” are Comparison Examples. It can clearly be seen that only the wax mixtures according to the invention show adequate viscosity stability or phase stability over the monitoring period.

### Test Series 1

Determination of the buildup of viscosity as a function of the ratio between the waxes used; ionic emulsifier.
Emulsifier: Sodium Stearoyl Glutamate (monosalt)—anionic;
Emollient: Cococaprylate/caprate—medium polarity

TABLE 1

| Ingredients | C1 | C2 | 1 | 2 | 3 | C3 | C4 |
|---|---|---|---|---|---|---|---|
| Cutina ® GMS | 5 | 4.25 | 3.75 | 2.5 | 1.25 | 0.75 | — |
| Pentaerythrityldistearate¹⁾ | — | 0.75 | 1.25 | 2.5 | 3.75 | 4.25 | 5 |
| Cococaprylate/Caprate | 16 | 16 | 16 | 16 | 16 | 16 | 16 |
| Sodium Stearoyl Glutamate | 0.5 | 0.5 | 0.5 | 0.5 | 0.5 | 0.5 | 0.5 |
| Glcerol | 3 | 3 | 3 | 3 | 3 | 3 | 3 |
| Water | 75.5 | 75.5 | 75.5 | 75.5 | 75.5 | 75.5 | 75.5 |
| Viscosity in mPa · s after | 12000 | 13200 | 15200 | 26000 | 22400 | 14800 | 12400 |
| production | | | | | | | |
| Viscosity in mPa · s after 4 W | 8000 | 10400 | 14800 | 26800 | 22400 | 7600 | 7600 |
| Viscosity in mPa · s after 8 W | unstable | 8000 | 14800 | 26800 | 22400 | 7600 | 6000 |
| Viscosity in mPa · s after 12 W | unstable | 4400 | 14000 | 26000 | 22000 | 6000 | 4000 |

¹⁾Ester of pentaerythritol which is obtained by reaction of 1 mol pentaerythritol with ca. 2 mol of a fatty acid mixture of 42 to 48% by weight C16 fatty acid and 50 to 56% by weight C18 fatty acid (rest: ≦C14 fatty acids and >C18 fatty acids), has the following ester distribution: 12 to 19% by weight monoesters, (b) 25 to 35% by weight diesters, (c) 30 to 40% by weight triesters and (d) 6 to 11% by weight tetraesters, and contains less than 0.3% by weight C₁₇ fatty acid acyl groups.

This ester was used in all the other tests (cf. following Tables).

### Test Series 2

Determination of the buildup of viscosity as a function of the ratio between the waxes used: ionic emulsifier.
Emulsifier: Sodium Cetearyl Sulfate—anionic;
Emollient: Cococaprylate/caprate—medium polarity

TABLE 2

| Ingredients | C5 | C6 | 4 | 5 | 6 | C7 | C8 |
|---|---|---|---|---|---|---|---|
| Cutina ® GMS | 5 | 4.25 | 3.75 | 2.5 | 1.25 | 0.75 | — |
| Pentaerythrityldistearate¹⁾ | — | 0.75 | 1.25 | 2.5 | 3.75 | 4.25 | 5 |
| Cococaprylate/Caprate | 16 | 16 | 16 | 16 | 16 | 16 | 16 |
| Sodium Cetearyl Sulfate | 0.5 | 0.5 | 0.5 | 0.5 | 0.5 | 0.5 | 0.5 |
| Glcerol | 3 | 3 | 3 | 3 | 3 | 3 | 3 |
| Water | 75.5 | 75.5 | 75.5 | 75.5 | 75.5 | 75.5 | 75.5 |
| Viscosity in mPa · s after | 12800 | 14000 | 18400 | 26000 | 23600 | 19600 | 18400 |
| production | | | | | | | |
| Viscosity in mPa · s after 4 W | 9600 | 10000 | 184000 | 26400 | 23200 | 10800 | 11200 |
| Viscosity in mPa · s after 8 W | 8000 | 8400 | 18000 | 26800 | 23600 | 10200 | 10800 |
| Viscosity in mPa · s after 12 W | unstable | unstable | 19200 | 26400 | 23600 | 8000 | 8000 |

### Test Series 3

Determination of the buildup of viscosity as a function of the ratio between the waxes used: ionic emulsifier.
Emulsifier: Sodium Stearate—anionic;
Emollient: Cococaprylate/caprate—medium polarity

TABLE 3

| Ingredients | C9 | C10 | 7 | 8 | 9 | C11 | C12 |
|---|---|---|---|---|---|---|---|
| Cutina ® GMS | 5 | 4.25 | 3.75 | 2.5 | 1.25 | 0.75 | — |
| Pentaerythrityldistearate¹⁾ | — | 0.75 | 1.25 | 2.5 | 3.75 | 4.25 | 5 |
| Cococaprylate/Caprate | 16 | 16 | 16 | 16 | 16 | 16 | 16 |
| Sodium Stearate | 0.5 | 0.5 | 0.5 | 0.5 | 0.5 | 0.5 | 0.5 |
| Glycerol | 3 | 3 | 3 | 3 | 3 | 3 | 3 |
| Water | 75.5 | 75.5 | 75.5 | 75.5 | 75.5 | 75.5 | 75.5 |
| Viscosity in mPa · s after | 14000 | 14000 | 18000 | 15200 | 10800 | 11200 | 12800 |
| production | | | | | | | |
| Viscosity in mPa · s after 4 W | 9200 | 9200 | 18400 | 15600 | 10200 | 6800 | 7200 |
| Viscosity in mPa · s after 8 W | unstable | 8000 | 18000 | 16000 | 10400 | 6000 | 6800 |
| Viscosity in mPa · s after 12 W | unstable | unstable | 18000 | 16000 | 10800 | 5600 | 6400 |

### Test Series 4

Determination of the buildup of viscosity in a 1:1 wax mixture of Cutina® GMS and pentaerythrityl distearate¹) as a function of the polarity of the emollients used: ionic emulsifier.
Emulsifier: Sodium Stearoyl Glutamate—anionic;
Emollient: Dicaprylylether—weak polarity; soybean oil—strong polarity

TABLE 4

| Ingredients | C13 | C14 | 10 | C15 | C16 | 11 |
|---|---|---|---|---|---|---|
| Cutina ® GMS | 5 | | 2.5 | 5 | | 2.5 |
| Pentaerythrityldistearate¹⁾ | — | 5 | 2.5 | | 5 | 2.5 |
| Dicaprylylether | 16 | 16 | 16 | | | |
| Soybean oil | | | | 16 | 16 | 16 |
| Sodium Stearoyl Glutamate | 0.5 | 0.5 | 0.5 | 0.5 | 0.5 | 0.5 |
| Glycerol | 3 | 3 | 3 | 3 | 3 | 3 |
| Water | 75.5 | 75.5 | 75.5 | 75.5 | 75.5 | 75.5 |
| Viscosity in mPa · s after | 3600 | 12400 | 21600 | 7200 | 14800 | 26800 |
| production | | | | | | |
| Viscosity in mPa · s after 4 W | <400 | 16600 | 22000 | 5200 | 18800 | 26800 |
| Viscosity in mPa · s after 8 W | unstable | 8000 | 20000 | unstable | 17200 | 27200 |
| Viscosity in mPa · s after 12 W | unstable | 6000 | 18800 | unstable | 12000 | 27200 |

### Test Series 5

Determination of the buildup of viscosity as a function of the ratio between the waxes used: nonionic emulsifier.
Emulsifier: Ceteareth-20—nonionic;
Emollient: Cococaprylate/caprate—medium polarity

TABLE 5

| Ingredients | C17 | C18 | 12 | 13 | 14 | C19 | C20 |
|---|---|---|---|---|---|---|---|
| Cutina ® GMS | 5 | 4.25 | 3.75 | 2.5 | 1.25 | 0.75 | — |
| Pentaerythrityldistearate¹⁾ | — | 0.75 | 1.25 | 2.5 | 3.75 | 4.25 | 5 |
| Cococaprylate/Caprate | 16 | 16 | 16 | 16 | 16 | 16 | 16 |
| Ceteareth-20 | 1 | 1 | 1 | 1 | 1 | 1 | 1 |
| Glycerol | 3 | 3 | 3 | 3 | 3 | 3 | 3 |
| Water | 75 | 75 | 75 | 75 | 75 | 75 | 75 |
| Viscosity in mPa · s after | 800 | 4400 | 9600 | 13200 | 13200 | 10000 | 9600 |
| production | | | | | | | |
| Viscosity in mPa · s after 4 W | 1600 | 1200 | 8800 | 14000 | 14000 | 4000 | 16400 |
| Viscosity in mPa · s after 8 W | unstable | 1200 | 8400 | 15600 | 13600 | 3600 | 10000 |
| Viscosity in mPa · s after 12 W | unstable | unstable | 8400 | 14400 | 14400 | 3600 | 10000 |

### Test Series 6

Determination of the buildup of viscosity as a function of the ratio between the waxes used: nonionic emulsifier.
Emulsifier: Lauryl glucoside—nonionic;
Emollient: Cococaprylate/caprate—medium polarity

TABLE 6

| Ingredients | C21 | C22 | 15 | 16 | 17 | C23 | C24 |
|---|---|---|---|---|---|---|---|
| Cutina ® GMS | 5 | 4.25 | 3.75 | 2.5 | 1.25 | 0.75 | — |
| Pentaerythrityldistearate¹⁾ | — | 0.75 | 1.25 | 2.5 | 3.75 | 4.25 | 5 |
| Cococaprylate/Caprate | 16 | 16 | 16 | 16 | 16 | 16 | 16 |
| Lauryl Glucoside (% active | 1 | 1 | 1 | 1 | 1 | 1 | 1 |
| substance) | | | | | | | |
| Glycerol | 3 | 3 | 3 | 3 | 3 | 3 | 3 |
| Water | 75 | 75 | 75 | 75 | 75 | 75 | 75 |
| Viscosity in mPa · s after | 3200 | 4400 | 4400 | 5600 | 3200 | 2400 | 1200 |
| production | | | | | | | |
| Viscosity in mPa · s after 4 W | <400 | 1200 | 4000 | 6000 | 3200 | <400 | <400 |
| Viscosity in mPa · s after 8 W | unstable | unstable | 3600 | 6000 | 3200 | unstable | unstable |
| Viscosity in mPa · s after 12 W | unstable | unstable | 4000 | 6400 | 3600 | unstable | unstable |

### Test Series 7

Determination of the buildup of viscosity in a 1:1 wax mixture of Cutina® GMS and pentaerythrityl distearate¹) as a function of the polarity of the emollients used: nonionic emulsifier.
Emulsifier: Ceteareth-20—nonionic;
Emollient: Dicaprylylether—weak polarity; soybean oil—strong polarity

TABLE 7

| Ingredients | C25 | C26 | 18 | C27 | C29 | 19 |
|---|---|---|---|---|---|---|
| Cutina ® GMS | 5 | | 2.5 | 5 | | 2.5 |
| Pentaerythrityldistearate¹⁾ | — | 5 | 2.5 | | 5 | 2.5 |
| Dicaprylylether | 16 | 16 | 16 | | | |
| Soybean oil | | | | 16 | 16 | 16 |
| Ceteareth-20 | 1 | 1 | 1 | 1 | 1 | 1 |
| Glycerol | 3 | 3 | 3 | 3 | 3 | 3 |
| Water | 75 | 75 | 75 | 75 | 75 | 75 |
| Viscosity in mPa · s after | 1600 | 2400 | 12800 | 4400 | 4800 | 11200 |
| production | | | | | | |
| Viscosity in mPa · s after 4 W | <400 | <400 | 12400 | 1200 | 9200 | 11600 |
| Viscosity in mPa · s after 8 W | unstable | unstable | 13200 | unstable | 8000 | 12000 |
| Viscosity in mPa · s after 12 W | unstable | unstable | 12000 | unstable | 2000 | 11600 |

1. A water-free wax mixture containing: (a) at least one member selected from the group consisting of esters of pentaerythritol, dipentaerythritol, and tripentaerythritol and (b) at least one partial glyceride, the ratio by weight of (a) to (b) being from 1:3 to 3:1.

2. The wax mixture as claimed in claim 1, wherein, component (a) comprises a member selected from the group consisting of C₆₋₂₂ fatty acid esters of pentaerythritol, dipentaerythritol, tripentaerythritol or mixtures of these esters which have a melting point of at least 30° C.

3. The wax mixture as claimed in claim 1, wherein, component (a) is selected from the group of esters of pentaerythritol with a percentage content of (i) 5 to 35% by weight monoesters, (ii) 20 to 50% by weight diesters and (iii) 25 to 50% by weight triesters and optionally tetraesters.

4. The wax mixture as claimed in claim 1, wherein, the ester component (a) is an ester with a fatty acid mixture containing 40 to 50% by weight C₁₆ fatty acids and 45 to 55% by weight C₁₈ fatty acids.

5. The wax mixture as claimed in claim 1, wherein, the partial glyceride (b) comprises at least one member selected from the group of esterification products of glycerol or oligoglycerol with C₁₂₋₂₄ fatty acids.

6. The wax mixture as claimed in claim 1, wherein, the partial glyceride (b) comprises a palmitic and/or stearic acid ester of glycerol with a monoglyceride content of 30 to 65% by weight and a diglyceride content of 25 to 45% by weight.

7. The wax mixture as claimed in claim 6, wherein, the partial glyceride (b) contains residues of palmitic and stearic acid in a ratio by weight of 1:3 to 3:1.

8. The self-emulsifying base containing the mixture claimed in claim 1, further comprising at least one emulsifier (c) selected from the group consisting of anionic emulsifiers and nonionic emulsifiers with an HLB value of at least 10.

9. The self-emulsifying base as claimed in claim 8 containing:
(a) 20% to 60% by weight of at least one member selected from the group consisting of C₆₋₂₂ fatty acid esters of pentaerythritol, C₆₋₂₂ fatty acid esters of dipentaerythritol and C₆₋₂₂ fatty acid esters of tripentaerythritol which have a melting point of at least 30° C.;
(b) 20% to 60% by weight of at least one partial glyceride selected from the group consisting of esterification products of glycerol with C₁₂₋₂₄ fat acids and esterification products of oligoglycerol with C₁₂₋₂₄ fatty acids; and
(c) 10% to 30% by weight of at least one additional emulsifier selected from the group consisting of anionic emulsifiers and nonionic emulsifiers with an HLB value above 10,
the self-emulsifying mixture containing less than 20% by weight water.

10. The self-emulsifying base as claimed in claim 8, wherein, the anionic emulsifier comprises at least one member selected from the group consisting of alkali metal salts of C₁₂₋₂₄ acyl glutamates, alkali metal and triethanolamine salts of C₁₂₋₂₄ fatty acids, and alkali metal salts of C₁₂₋₂₄ fatty alcohol (ether) sulfates and C₁₂₋₂₄ fatty alcohol (ether) phosphates.

11. The self-emulsifying base as claimed in claim 9, wherein, the nonionic emulsifiers with an HLB value of at least 10 are selected from the group of C₁₂₋₂₄ fatty alcohol ethoxylates and/or C₁₂₋₂₄ alk(en)yl oligoglycosides.

12. The cosmetic or pharmaceutical preparation containing at least one member selected from the group consisting of 1% to 20% by weight of the wax mixture of claim 1 and 1% to 20% by weight of a self-emulsifying base comprising the wax mixture and at least one of a nonionic emulsifier with an HLB value of at least 10 and an anionic emulsifier with an HLB value of at least 10.

13. An oil-in-water emulsion containing the wax mixture claimed in claim 1 whereby the viscosity of the emulsion is increased.

14. An oil-in-water emulsion containing the self-emulsifying mixture claimed in claim 8, whereby, the viscosity of the emulsion is increased.

15. The cosmetic or pharmaceutical emulsion containing the wax mixture claimed in claim 1 as a consistency factor.

16. The cosmetic or pharmaceutical emulsion containing the self-emulsifying mixture of claim 8 as a consistency factor.

17. The wax mixture as claimed in claim 2, wherein, component (a) is selected from the group of esters of pentaerythritol with a percentage content of (i) 5 to 35% by weight monoesters, (ii) 20 to 50% by weight diesters and (iii) 25 to 50% by weight triesters and optionally tetraesters.

18. The wax mixture as claimed in claim 2, wherein, the ester component (a) is an ester with a fatty acid mixture containing 40 to 50% by weight C₁₆ fatty acids and 45 to 55% by weight C₁₈ fatty acids.

19. The wax mixture as claimed in claim 2, wherein, the partial glyceride (b) comprises at least one member selected from the group of esterification products of glycerol or oligoglycerol with C₁₂₋₂₄ fatty acids.

20. The self-emulsifying base containing the mixture claimed in claim 2, further comprising at least one emulsifier (c) selected from the group consisting of anionic emulsifiers with an HLB value of at least 10 and nonionic emulsifiers with an HLB value of at least 10.
